(19) United States
(12) Patent Application Publication
(10) Pub. No.: US 2021/0299276 A1
(43) Pub. Date: Sep. 30, 2021

(54) METHODS OF EXPLOITING ONCOGENIC DRIVERS ALONG THE HUMAN CYCLIN G1 PATHWAY FOR CANCER GENE THERAPY

(71) Applicants: Erlinda M. Gordon, Santa Monica, CA (US); Frederick L. Hall, Carmel Valley, CA (US)

(72) Inventors: Erlinda M. Gordon, Santa Monica, CA (US); Frederick L. Hall, Carmel Valley, CA (US)

(21) Appl. No.: 17/346,089

(22) Filed: Jun. 11, 2021

Related U.S. Application Data
(63) Continuation of application No. PCT/US2019/066392, filed on Dec. 13, 2019.
(60) Provisional application No. 62/779,278, filed on Dec. 13, 2018.

(51) Int. Cl. A61K 48/00; A61K 31/45; A61K 31/496; A61K 31/167; A61K 31/4745; A61K 31/519; A61K 31/704; A61K 31/4995; A61K 39/395; A61K 9/00; A61K 31/357; A61K 31/05; A61K 31/19; A61P 35/00; G01N 33/50

(52) U.S. Cl. CPC A61K 48/005; G01N 33/5011; A61K 31/496; A61K 31/167; A61K 31/4745; A61K 31/519; A61K 31/704; A61K 31/4995; A61K 39/3955; A61K 9/0019; A61K 9/0014; A61K 31/357; A61K 31/05; A61K 31/19; A61P 35/00; A61K 31/45

(57) ABSTRACT

The present disclosure teaches methods of treating a patient having an advanced metastatic cancer by administering a plurality of infusions of a first therapeutic agent comprising a tumor-targeted gene vector that encodes a cytocidal inhibitor of the CCNG1 gene product and a second therapeutic agent that affects the activity of at least one additional molecular target along the CCNG1 pathway. The additional molecular target may be Mdm2, PP2A, p53, Rb, c-Myc, or a cyclin-dependent kinase. The present disclosure also provides methods of treatment by administering a plurality of infusions of a first therapeutic agent comprising a tumor-targeted gene vector that encodes a cytocidal inhibitor of the CCNG1 gene product and a second therapeutic agent such as an immune-modulatory monoclonal antibody, a cytotoxic chemotherapeutic agent, an anti-angiogenesis agent, a selective tyrosine kinase inhibitor, or a monoclonal antibody directed against specific features of cells from the metastatic cancer. Further, the disclosure provides methods of treating a palpable tumor, methods for evaluating the role of oncogenic drivers along the Cyclin G1 pathway in a tumor, and methods of treatment that use such evaluations/analyses to guide the management of the disease.

### CROSS-REFERENCE TO RELATED PATENT APPLICATIONS

This application is a continuation of the International Application PCT/US2019/066392 filed on Dec. 13, 2019, which claims the benefit of U.S. Provisional Application No. 62/779,278 filed on Dec. 13, 2018, each of which are hereby incorporated by reference in their entireties.

### TECHNICAL FIELD

The present disclosure relates generally to methods of treating a patient with cancer. More specifically, this disclosure relates to methods for exploiting the pivotal and therapeutically accessible locus of executive Cell Cycle Checkpoint Control—focusing specifically on the Cyclin G1 protein (CCNG1 proto-oncogene-product) and the associated oncogenic drivers arrayed along the aberrant biochemical pathways that (i) promote and (ii) ensure uncontrolled cell proliferation, resulting in oncogenesis, increasingly aggressive metastasis, and chemotherapeutic refractoriness.

### BACKGROUND

Molecular Drivers of the Cell Division Cycle: Overriding Normal Cell Cycle Checkpoint Control. Basic research into the primal executive mechanisms governing the cell division cycle have identified site-specific (primary sequence specific) protein phosphorylation to be a major regulatory theme that governs the transition phases of the cell cycle—that is, the orderly “activation” of quiescent stem cells at the G0 to G1 boundary to become “capable” of cell proliferation (Competence Promoting Factor); followed by the initiation of DNA synthesis (S-phase Promoting Factor); followed by genomic proofreading and the physical partitioning into daughter cells via the elegant biomechanics of mitosis (M-phase Promoting Factor). Conceptually, this family of executive cell cycle control enzymes are site-specific protein kinases (phosphotransferases; a.k.a. cyclin-dependent protein kinases or CDKs), which recognize specific structural features, or target sequences characterized by, e.g., Hall & Vullietl, arrayed along major cell-cycle and gene-regulatory proteins. Thus, a canonical “Cyclin” is thought of as an oscillating, positive-acting “regulatory subunit” of an identified CDK, which both “activates” the catalytic subunit and physically “targets” the otherwise inactive and otherwise undiscerning (blind) kinase to the cognate phosphorylation site(s) of the targeted cell cycle regulatory proteins.

Named alphabetically in order of discovery/molecular characterization, the so-called canonical “Cyclins” are positive-acting regulatory targeting subunits of the CDK holoenzymes that are periodically expressed, assembled, activated, and catabolized, in strict accordance with the discrete phases of the cell division cycle. Working backwards in cell-time, from the massive global kinase activity associated with mitosis (M-phase Promoting Factor=Cyclin B+CDCl₂), to the decisive executor/tumor suppressor functions associated with S-phase entry (S-phase Promoting Factor=Cyclin A+CDK2, or CDCl₂), to the heightened metabolic pathways associated with sustained G1 or growth phase (Cyclins D1, D2, D3, E+respective kinase partners): the identification of Cyclin G1 (CCNG1 proto-oncogene), an early riser, and the assessment of its constitutive expression, as well as its essential function, in human cancer cells, represented somewhat of a conundrum at the time: it looked very much like a canonical “Cyclin” protein, in terms of primary structure of its telltale “Cyclin Box” (see FIG. 1); while (i) it lacked an identifiable kinase partner, (ii) it lacked an identifiable phospho-acceptor “target protein,” and (iii) it lacked the cyclical behavior of the so-called “canonical Cyclins” that agreeably and accordingly marked the major phase transitions of the mammalian cell division cycle.

Fortunately, what was hidden from the wise in terms of rigid canonical considerations, was revealed to the experimentalists and physician-scientists, who looked beyond the meager definitions to explore the actual structure/function relations of the Cyclin G1 protein (by genetic engineering of CCNG1) in the context of cancer gene therapy, long before Cyclin G1 was determined to be the prime molecular driver of the elusive Cell Competence Factor, the pivotal executive component of the Cyclin G1/p53/Mdm2 Axis governing cell cycle checkpoint control, and perhaps, a most strategic target for new precision molecular and genetic cancer therapies, as well as chemo-sensitization¹.

Biochemically, Cyclin G1 (CCNG1 gene product), was a “non-canonical” yet demonstrably essential and potentially oncogenic “Cyclin-like” protein, whose first appearance (expression) and executive action is one of the earliest cell cycle events that drive the quiescent stem cell from G0, to enter G1 phase. Mechanistically, Cyclin G1 physically binds to a major cellular ser/thr protein phosphatase subunit designated 2A (PP2A), thereby “targeting” the otherwise undiscerning phosphatase activity to a Cyclin G1-targeted protein, which happens to be Mdm2 (oncogene product)—the Mdm2 protein, in turn, targets, inhibits, and degrades the p53 tumor suppressor, an often-lost, yet vitally important substrate (guardian of DNA fidelity with executioner functions) in the normal regulation of the cell division cycle. This oncogenic pathway (i.e., the Cyclin G1/Mdm2/p53 Axis) is distinguishable from the set of canonical oncogenic G1 cyclins (D1,2,3, Cyclin E, Cyclin A) that target CDK complexes cyclically and precisely to pRB (and Rb-related) tumor suppressor proteins, whose inhibition releases E2F transcription factors that drive cells to irreversibly enter the S-phase of the division cycle (G1 to S) (Cyclin/CDK/Rb/E2F Axis)¹.

The biochemical “activation” of the Mdm2 oncoprotein by the oncogenic Cyclin G1 is a crucial link in the emerging Cyclin G1/Mdm2/p53 Axis. The Mdm2 gene itself is amplified/over-expressed in numerous human cancers including soft tissue sarcoma, osteosarcoma and esophageal carcinoma^{2,3}. The Mdm2 oncoprotein is known to form a physical complex with the p53 tumor suppressor, thus, inhibiting its transcriptional “tumor suppressor” function; in addition, Mdm2 acts as a specific E3 ubiquitin ligase, responsible for the ubiquitination and ultimate degradation of the p53 tumor suppressor protein⁴. Thus, the oncogenic potential of Mdm2 to override the decisive/protective tumor-suppressive functions of wild-type p53 is uniquely, if not entirely, Cyclin G1-dependent. Additional support for the executive role of Cyclin G1—in relation to the pivotal Cyclin G1/Mdm2/p53 Axis—came from high-throughput screening for regulatory micro-RNA species that are commonly lost with the development of human cancers: Apparently, the major species (˜70% of the total population) of regulatory micro-RNAs that are commonly lost in the pathogenesis and stage-wise progression of hepatocellular carcinoma is miR-122, which physically “targets” the CCNG1 (Cyclin G1) gene for suppression, and thus appears to be a natural growth suppressive-microRNA focused on limiting the expression of Cyclin-G1 in the quiescent stem cells of this potentially proliferative, highly regenerative organ. Turning to virology, a renewed appreciation of the oncogenic potential of dysregulated CCNG1 gene expression—in terms of both persistent stem cell activation (Cell Competence) and over-riding p53-mediated checkpoint control (thus driving Cell Survival over DNA fidelity)—came to light when it was discovered that the carcinogenic hepatitis B virus (HBV) produces a protein, the HBx-protein, which specifically, directly, perhaps strategically, down-regulates the normal expression of miR-122⁶, which results in increased CCNG1 gene expression; raising Cyclin G1 to sufficient levels that Cyclin-G/PP2A complexes activate Mdm2 and ultimately over-ride the executor/suppressor functions of wild-type p53, thereby abolishing the well-known p53-mediated inhibition of HBV replication, as well⁶.

Finally, the curiously non-canonical Cyclin G1 was formally ushered (at least experimentally) into the ballroom, with the “Grand-Prize of the Fair” as its cellular target: that is, the illustrious c-Myc oncogene, long considered to be the most “desirable” molecular locus for clinical intervention in all of cancer therapy; and yet it was—until now—considered to be among the “least druggable” of all the cancer targets^{1,7}. The recent discovery that the once-non-canonical Cyclin G1 partners with CDK2 (and CDK5, on occasion) to physically target and site-specifically phosphorylate (activate) the c-Myc oncoprotein, which in turn, provides the transcriptional drive for selective protein synthesis at the very threshold of the G0 to G1 transition, is both informative and important: in that this newfound Cyclin G1/CDk2/c-Myc Axis of stem cell activation represents the necessary biochemical linkage to the theoretical (Competence Promoting Factor), which appears whenever quiescent stem cells regain Cell Competence—competence to proliferate as needed for normal tissue repair; and when it comes to cancer, competence to proliferate continuously. It is in this remarkable association with c-Myc that the biochemical contingencies for canonization of Cyclin G1 are finally met: (i) Cyclin G1 gains two attractive CDK partner(s); (ii) Cyclin G1 gains a critical substrate target protein, the elusive c-Myc oncoprotein, and (iii) the absence of cyclical oscillations in the levels of Cyclin G1 protein expressed in cancer cells, in cadence with the discrete phases cell division cycle, is readily explainable by the provocative notion that cancer cells are constitutively Competent in terms of this first-and-rate-limiting oncogenic Cyclin driver, CCNG1. The clinical upside of this provocative notion, is that the strategic blockade of Cyclin-G1 function, by experimental suppression of CCNG1 gene expression or the molecular blockade of Cyclin-G1-dependent pathways (FIG. 1), is invariably fatal to the cancer cell, thus clinically effective, in a number of human cancers¹.

Mechanistically, c-Myc is a critical PDGF-inducible ‘Competence gene’ that activates diverse cellular processes associated with entry into and progression through the cell cycle, including the synthesis of cellular components in preparation for growth, DNA synthesis, and cell division. It is in this manner, by activating and selectively targeting Cdk5 kinase activity to activate and stabilize the c-Myc oncoprotein, that the overexpression of CCNG1 enables cancer cells to overcome radiation-induced (i.e., DNA-damage-induced) cell cycle arrest^{1,7}. Although the transcriptional targets of c-Myc include a number of DNA repair genes, thereby coupling DNA replication to the pathways and processes that preserve the integrity of the genome, the net effect of CCNG1 function in association with Cdk5 (or Cdk2) is to abrogate DNA-fidelity checkpoint controls to promote Cell Survival, Cell Competence, and Cell Cycle Progression at the ‘peril’ of increasing error-prone DNA synthesis, as is often found in cancers.

Cyclin G1 Pathway Inhibitor Therapy: Genetic Engineering of a Killer Gene Product. The first tumor-targeted gene therapy product that is based on the strategic blockade of Cyclin G1-dependent pathways is DeltaRex-G (former names: Rexin-G, Mx-dnG1), which encodes a dominant negative mutant construct of the CCNG1 gene (designated dnG1 protein) that is devoid of the ubiquitinated N-terminus (proteolytic processing), as well as the first two helical segments (a1 and a2) of the definitive cyclin box, characteristically arrayed in “Cyclins” as a tandem set of helical segments, including two highly-conserved residues essential for cyclin-dependent kinase (CDK) binding¹. The cytocidal dnG1 protein, which induces apoptosis in proliferative cells, retains the presumptive CDK contact points (Helix α3*, α5*) and the complete structural domains attributed to PP2A, pi and Mdm2 binding. Recently, new therapeutic synthetic peptides (e.g., ELAS1 and a5 Helix peptides, see 1) derived from structures and/or homologous interfaces contained within the dnG1 protein are themselves reported to induce cell cycle blockade and apoptosis, respectively (FIG. 1). Suppressing CCNG1 expression with miR-122, as well as CCNG1 silencing, increases the sensitivity of HCC cells to doxorubicin⁹, thereby establishing the rational basis for of combined gene- chemo- and miRNA-based therapies for HCC, based on the suppression/blockade of Cyclin G1-dependent pathways.

Meanwhile, there is increasing clinical evidence that innovative tumor-targeted cancer therapies—based on the progress made in discovering, characterizing, and elaborating the structure/function relationships of Cyclin G1—may indeed be uniquely effective in managing aggressive metastatic cancers, such that repeated infusions of DeltaRex-G, a tumor-targeted dnG1 expression construct, were determined to be potentially curative, even when standard chemotherapies had previously failed¹. In addition to statistically significant gains in patient overall survival, a considerable number of advanced-stage, chemotherapy-resistant cancer patients treated with repeated infusions of DeltaRex-G as monotherapy (i.e., single-agent efficacy): including metastatic pancreatic cancer, osteosarcoma and soft tissue sarcoma patients, remain cancer-free or without active disease progression 10 years after the initiation of DeltaRex-G treatment¹⁰. Table 1 lists and summarizes the results of 5 U.S. based Phase 1/2 clinical trials and one Phase 2 study with long term survivors, that have resulted in orphan drug designations of DeltaRex-G for pancreatic cancer, soft tissue sarcoma and osteosarcoma, and fast track designation of DeltaRex-G for pancreatic cancer¹¹⁻¹⁷. Hence, the development of DeltaRex-G, which by itself, induces apoptosis in cancer cells and tumor-associated vasculature (in the presence or absence of a functional p53 gatekeeper), may be a powerful new clinical application in terms of applied cell cycle checkpoint control, which merits conscientious clinical development. Our ongoing studies have confirmed that CCNG1 expression is predictably elevated in many types of cancers″, which suggests that monitoring CCNG1 expression in tumors, as well as its associated oncogenic effectors, may identify patients who will benefit from CCNG1 inhibitor therapy.

Perspectives on New Combinatorial Approaches to Cancer Management: Targeted cancer therapies are likely to be more effective and less toxic to normal cells than standard chemotherapeutic agents and radiation therapy¹⁹. These therapies are commonly used alone, in combination with other targeted therapies, and in combination with other cancer treatments such as chemotherapy. Targeted cancer therapies approved for clinical use include drugs that block cell growth signaling (e.g., tyrosine kinase and serine-threonine kinase inhibitors), drugs that either disrupt tumor blood vessel development (e.g., bevacizumab), evoke apoptosis or programed death of specific cancer cells (e.g., trabectedin), activate the immune system to recognize tumor neoantigens and destroy specific cancer cells (e.g., cancer vaccines and immune checkpoint inhibitors), and/or deliver cytotoxic toxic drugs (e.g. nab-paclitaxel) to cancer cells. Based on observations and reports of chemo-sensitization, we theorize that combinatorial therapies using DeltaRex-G, a Cyclin G1 inhibitor and other molecular targets along the CCNG1 pathway, including Mdm2, PP2A, p53, Rb and c-Myc, may exert additive, complementary, and/or synergistic effects in the treatment of advanced metastatic cancers. Drugs which are already FDA approved, or are currently in clinical trials, include the following: the Mdm2 inhibitor (e.g. AMG232 and Nutlin 3a)²⁰, the CDK4/CDK6/Rb inhibitor Palbociclib (PD0332991, Ibrance)²¹, and the mutated p53 inhibitor SAHA (Vorinostat)²². While c-Myc is overexpressed in many kinds of cancer, strategies to effectively modulate c-Myc activity (outside of modulating its targeting/activation by Cyclin G1) do not yet exist; however, the small molecule anticancer agent APTO-253 appears to inhibit c-Myc expression to some degree, while inducing cell cycle arrest and apoptosis in certain hematologic malignancies²³.

It is possible that repeated Cyclin G1 inhibition alone, such as by DeltaRex-G—by suppressive genetic strategies and/or selective biochemical blockades—will turn out to be a necessary and sufficient treatment regimen, in terms of cancer gene therapy. However, at this reflective point in time, it would also be prudent to monitor the associated pharmacological effects on the other major oncogenic drivers within these newly characterized Cyclin-G1 dependent pathways. Evaluating the safety and efficacy of DeltaRex-G combination regimens: that being Cyclin G1 inhibitor therapy, combined with modulating one or more of the executor proteins (CDK2/5, PP2A, p53, c-Myc) in the CCNG1 pathway represents a new opportunity for advancement of cell cycle checkpoint inhibitors in the field of cancer medicine. On the other hand, DeltaRex-G is cytocidal to cancer cells, tumor associated vasculature and malignant stromal fibroblasts and may well prime the recruitment and/or entry of cytokines, immune modulators²⁴⁻²⁶, and potentially, chemotherapeutic, anti-angiogenic and targeted therapies into the tumor microenvironment. For instance, in a Phase 1/2 study using DeltaRex-G+Reximmune-C, a tumor-targeted gene vector encoding a human GM-CSF gene, the reported one-year overall survival was 86% in chemo-resistant solid tumors and B-cell lymphoma²⁵′²⁶. Hence, combinatorial therapies external to the CCNG1 inhibitor pathway may include DeltaRex-G plus (i) immune-modulatory monoclonal antibodies, including FDA-approved immune checkpoint inhibitors, (ii) cytotoxic chemotherapies such as doxorubicin and trabectedin, (iii) anti-angiogenesis agents such as bevacizumab, (iv) selective tyrosine kinase inhibitors, and/or (v) monoclonal antibodies directed against specific features of the evolving metastatic cancer cells (e.g. panitumumab, cetuximab). Viewed from a biochemical perspective, which teaches that “the-first-and-rate-limiting-step” of a given biochemical pathway is often the most important, as it is often leveraged, in terms of regulatory cause-and-effect, the strategic blockade of Cyclin G1 function—its Competence-Promoting function and its Pro-Survival function in the face of increasing genetic instability—sets the stage for the new clinical applications and optimization of combinatorial therapies with renewed assurance that Cyclin G1 itself is a strategic therapeutic locus indeed.

The current disclosure is supported by the following lines of reasoning: (i) a critical analysis of the tumor responses of patients currently being treated with DeltaRex-G and oncogene suppressors (ii) analysis of the objective “immunological’ tumor responses in Stage 4 pancreatic cancer, B-cell lymphoma in light of the latest clinical long-term survival data (>10 years cancer free); (iii) the notorious lack of appreciable anticancer immunity (and/or associated adoptive immune responses) seen in Stage 4 pancreatic cancer patients, and (4) the natural conversion of B cell lymphoma, otherwise non-immunogenic, into an immunogenic phenotype and long term survival with DeltaRex-G followed by DeltaVax (a tumor-targeted gene vector encoding GM-CSF) therapy.

### BRIEF DESCRIPTION OF THE DRAWINGS

The embodiments disclosed herein will become more fully apparent from the following description and appended claims, taken in conjunction with the accompany drawings.

FIG. 1 Structure/Function analysis of the executive Cyclin G1 gene product. Left Panel: Cyclin G1 Functional Domains. Cyclin G1 physically binds to the ser/thr protein phosphatase subunit designated 2A (PP2A) to activate a key regulatory oncoprotein, Mdm2. The Mdm2 oncoprotein forms a physical complex with the p53 tumor suppressor, thus, inactivating its tumor suppressor function, while additionally acting as a specific E3 ubiquitin ligase that is responsible for the ubiquitination and degradation of the p53 tumor suppressor protein (4). This dephosphorylation event is Cyclin G1-dependent. Cyclin G1 also activates CDK5 and CDK2 to target/activate the c-Myc onco-protein. Right Panel: dnG1 Dominant Negative G1 (Killer Gene). The experimentally optimized Cyclin G1 inhibitor, a cytocidal a dominant-negative mutant construct of Cyclin G1, is devoid of the ‘ubiquitinated’ N-terminus (proteolytic processing), as well as the first two helical segments (α1 and α2) of the definitive Cyclin Box: characteristically arrayed in cyclins as a tandem set of helical segments, including two highly-conserved residues (asterisks) essential for Cyclin-dependent kinase (Cdk) binding. The cytocidal dnG1 protein—which induces apoptosis in proliferative cells—retains the presumptive CDK contact points (Helix α3*, α5*) and the structural domains attributed to PP2A, β′ and Mdm2 binding. Remarkably, small synthetic peptides (e.g., ELAS1 and 5 Helix peptides) derived from structures or homologous interfaces contained within the cytocidal dnG1 protein have been reported to induce cell cycle blockade and apoptosis, respectively.

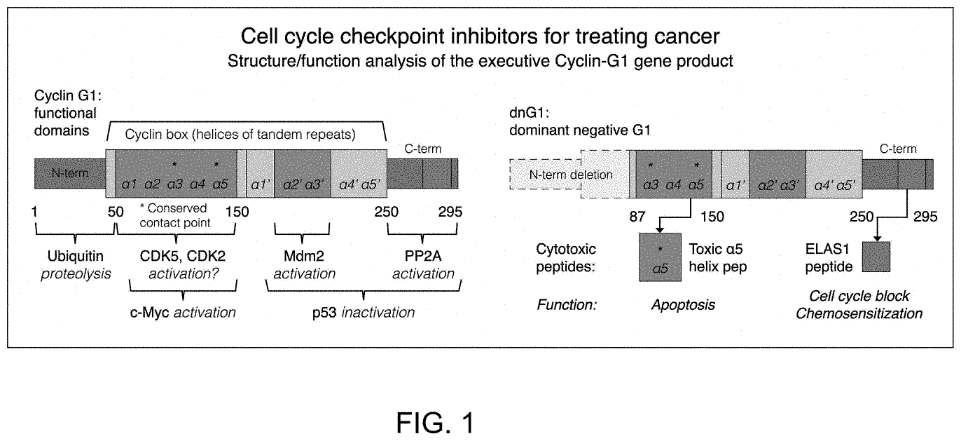

FIG. 2 Reduction in size of palpable sarcomas during DeltaRex-G treatment and transdermal delivery of oncogene suppressor balms. A: Before treatment—Day 1: Patient with clear cell sarcoma was treated with SD-Sarc-Balm 1, applied topically, generously over the tumor nodule. After 2 hours, Sarc-Balm 2 was applied topically, slowly, generously over tumor nodule. Applications were repeated twice a day for 3 days. B: After treatment—Day 3: Dramatic reduction in the size of the subcutaneous tumor.

### DETAILED DESCRIPTION

The present disclosure provides methods of treating cancer. Such methods may comprise multiple infusions of a tumor targeted gene vector encoding a cytocidal inhibitor of the CCNG1 gene product in combination with other molecular targets along the CCNG1 pathway, including Mdm2, PP2A, p53, Rb and c-Myc, which may exert additive, complementary, and/or synergistic effects in the treatment of advanced metastatic cancers. Drugs which are already FDA approved, or are currently in clinical trials, include the following: the Mdm2 inhibitor (e.g. AMG232 and Nutlin 3a)²⁰, the CDK4/CDK6/Rb inhibitor Palbociclib (PD0332991, Ibrance)²¹, and the mutated p53 inhibitor SAHA (vorinostat)²². While c-Myc is overexpressed in many kinds of cancer, strategies to effectively modulate c-Myc activity (outside of modulating its targeting/activation by Cyclin G1) do not yet exist in clinical medicine; however, the small molecule anticancer agent APTO-253 appears to inhibit c-Myc expression to some degree, while inducing cell cycle arrest and apoptosis in certain hematologic malignancies²³.

In certain embodiments, the methods of treating a patient having an advanced metastatic cancer comprise (1) administering to the patient a plurality of infusions of a first therapeutic agent comprising a tumor-targeted gene vector that encodes a cytocidal inhibitor of the CCNG1 gene product; and (2) administering to the patient a second therapeutic agent that affects the activity of at least one additional molecular target along the CCNG1 pathway. The administrations of the first and second therapeutic agents are determined according to the pharmacological properties of each therapeutic agent. As a result, the first and second therapeutic agents may be administered at the same time or at different times. Likewise, the number of administrations of the therapeutic agents may be the same or they may be different. In some embodiments, the first therapeutic agent comprises DeltaRex-G.

The words “treating” and “treatment” have their usual meanings in medical science, that is, “treating” means the management and care of a patient to cure or alleviate a disease or disorder or the symptoms thereof. A treatment may achieve a “cure,” that is, a complete and permanent remission of a cancer, but it need not be a cure. Treatment may be undertaken to alleviate symptoms, for example, to decrease tumor size, the number and location of metastases, or the physiological effects of tumor burden. Treatment may lead to temporary remission or render the tumor more amenable to other therapeutic options (such as surgery, radiation, or treatment with a different therapeutic agent or combination of agents). It should also be noted that use of these terms is not meant to exclude other steps that may be necessary or desirable for the management and care of a cancer patient but that are not recited in the methods described in this disclosure, e.g., use of IV fluids for the patient's hydration or use of medications to treat pain.

In some embodiments, the additional molecular target may be one or more of the Mdm2, PP2A, p53, Rb and c-Myc gene products. When the additional molecular target is an Mdm2 gene product, the second therapeutic agent may be AMG232, Nutlin 3a, or other Mdm2 inhibitor. When the additional molecular target is a p53 gene product, the second therapeutic agent may be an inhibitor of mutated p53, such as SAHA (vorinostat). When the additional molecular target is a c-Myc gene product, the second therapeutic agent may be APTO-253 or other c-Myc inhibitor.

In some embodiments, the additional molecular target may be one or more cyclin-dependent kinases. In a subset of such embodiments, the second therapeutic agent may be palbociclib.

Other techniques for affecting the activity of a molecular target are known in the art and may be used in the methods disclosed herein. For example, in certain embodiments in which the molecular target is a tumor suppressor gene product (for example, p53 or Rb), the second therapeutic agent may be an expression vector that encodes the tumor suppressor gene. This expression vector may be a tumor-targeted vector. In certain other embodiments, the second therapeutic agent modifies RNA levels. In such embodiments, the second therapeutic agent may be an antisense oligonucleotide, an RNAi construct, ribozyme, or other suitable agent.

The present disclosure also provides methods of treating a cancer in a patient in which multiple infusions of a tumor targeted gene vector encoding a cytocidal inhibitor of the CCNG1 gene product are administered in combination with (i) immune-modulatory monoclonal antibodies, including FDA-approved immune checkpoint inhibitors, (ii) cytotoxic chemotherapies such as doxorubicin and trabectedin, (iii) anti-angiogenesis agents such as bevacizumab, (iv) selective tyrosine kinase inhibitors, and/or (v) monoclonal antibodies directed against specific features of the evolving metastatic cancer cells (e.g. panitumumab, cetuximab). The cancer may be an advanced metastatic cancer.

In certain embodiments, the methods of treating a patient having an advanced metastatic cancer comprise (1) administering a plurality of infusions of a first therapeutic agent comprising a tumor-targeted gene vector that encodes a cytocidal inhibitor of the CCNG1 gene product; and (2) administering a second therapeutic agent that is selected from the group consisting of immune-modulatory monoclonal antibodies, cytotoxic chemotherapies, anti-angiogenesis agents, selective tyrosine kinase inhibitors, and monoclonal antibodies directed against specific features of cells from the metastatic cancer. In some embodiments, the first therapeutic agent comprises DeltaRex-G.

In certain embodiments, the second therapeutic agent comprises an immune-modulatory monoclonal antibody. In a subset of such embodiments, the second therapeutic agent may be one or more checkpoint inhibitors. In certain other embodiments, the second therapeutic agent comprises a cytotoxic chemotherapy agent. In a subset of such embodiments, the second therapeutic agent may be doxorubicin, trabectedin, other known chemotherapy agent, or combination thereof. In certain other embodiments, the second therapeutic agent comprises an anti-angiogenesis agent. In a subset of such embodiments, the second therapeutic agent may be bevacizumab. In certain other embodiments, the second therapeutic agent comprises a selective tyrosine kinase inhibitor. In certain other embodiments, the second therapeutic agent comprises one or more monoclonal antibodies directed against specific features of cells from the metastatic cancer. In a subset of such embodiments, the second therapeutic agent may be panitumumab, cetuximab, or a combination thereof.

The present disclosure further provides methods of treating palpable tumors. Such methods may comprise administering multiple infusions of a tumor targeted gene vector encoding a cytocidal inhibitor of the CCNG1 gene product in combination with transdermal delivery of well-characterized bioactive agents with well-defined biochemical mechanisms of action, followed by “Astute Analysis” of treatment/tumour response sensitivity to Apoptosis-Inducing-agents, which involve Oncogene suppression at the level of promoters (i.e., c-MYC and CCNG1 gene suppression) vis-a-vis Ferroptosis-Inducing-Agents, (eg Artemisinin, Trans-Resveratrol) which is a general and yet selective vulnerability of cancer cells based on the differential propensity to accumulate Iron (Fe+).^{27,31} The “Astute Analysis” involves the scientific understanding that a biochemical class of agents (complex phenolic compounds), specifically pentacyclic triterpines: including betulinic acid, oleanolic acid, and boswellic acid are potent inhibitors of oncogene expression (including c-Myc and CCNG1) at the genetic level, as well as inducing apoptosis and tumor regression on the histological level,²⁸⁻³⁰ which can be used together for diagnostic purposes, as described below.

In certain embodiments, the methods of treating a palpable tumor in a patient comprise: (1) administering a plurality of infusions of a first therapeutic agent comprising a tumor-targeted gene vector that encodes a cytocidal inhibitor of the CCNG1 gene product; followed by (2) transdermally administering a second therapeutic agent comprising a ferroptosis-inducing-agent; and (3) thereafter transdermally administering a third therapeutic agent comprising an apoptosis-inducing agent. In some embodiments, the first therapeutic agent comprises DeltaRex-G. The ferroptosis-inducing-agent may be Artemisinin, Trans-Resveratrol, or other suitable feeroptosis-inducing agent, or combinations thereof. The apoptosis-inducing agent may be a pentacyclic triterpine, e.g., betulinic acid, oleanolic acid, boswellic acid, or combinations thereof.

In certain embodiments, the third therapeutic agent is selected from a plurality of apoptosis-inducing agents after determining response sensitivity of the tumor, following administration of the first and second therapeutic agents, to the apoptosis-inducing agents and selecting at least one apoptosis-inducing agent to which the tumor is sensitive.

The present disclosure also provides methods of evaluating oncogenic drivers along the Cyclin G1 pathway histologically (eg, MDM2, TP53 CCNG1, MYC) to provide molecular diagnostic insights that may be combined with local non-invasive treatment/analysis for differential diagnoses of best treatment options and contraindications.

In certain embodiments, the methods for evaluating the role of oncogenic drivers along the Cyclin G1 pathway in a tumor in a patient comprise: (1) determining the sensitivity of the tumor to at least one bioactive agent selected from the group consisting of ferroptosis-inducing agents, apoptosis-inducing agents, and combinations thereof by (a) administering the bioactive agent to at least a portion of the tumor and (b) evaluating any effects on a characteristic of the tumor; wherein tumor sensitivity to the at least one bioactive agent indicates the operation of oncogenic drivers in the tumor. The tumor may be a sarcoma. A “characteristic” of the tumor may be tumor size, hardness, pain, etc. In other words, a decrease in size, hardness (e.g., a more fluctuant tumor), or pain indicates tumor sensitivity to the bioactive agent or agents. In some embodiments, the bioactive agent is a pentacyclic triterpine, including the examples discussed above.

In some embodiments, the tumor is palpable and the bioactive agents are administered transdermally. Such non-invasive methods provide helpful diagnostic insights that help guide the patient's treatment. The administration of the bioactive agent(s) may also decrease tumor size, which may be therapeutically beneficial.

As discussed above, these methods for evaluating the role of oncogenic drivers may help guide therapy. If, for example, tumor sensitivity to at least one bioactive agent may indicate the operation of oncogenic drivers in the tumor. Thus, in some embodiments, such methods comprise: (1) evaluating the role of oncogenic drivers along the Cyclin G1 pathway in the tumor as discussed above; and (2) if the tumor is sensitive to the at least one bioactive agent, administering a plurality of infusions of a first therapeutic agent comprising a tumor-targeted gene vector that encodes a cytocidal inhibitor of the CCNG1 gene product to the patient. In some embodiments, the first therapeutic agent comprises DeltaRex-G.

In certain embodiments, such methods may further comprise the step of administering a second therapeutic agent that affects the activity of at least one additional molecular target along the CCNG1 pathway. This additional molecular target may be one of the targets discussed above. The second therapeutic agent may be one of the agents discussed above.

It will be readily understood that the embodiments, as generally described herein, are exemplary. This detailed description of various embodiments is not intended to limit the scope of the present disclosure but is merely representative of various embodiments.

### EXAMPLES

The following examples are illustrative of disclosed methods and compositions. In light of this disclosure, those of skill in the art will recognize that variations of these examples would be possible without undue experimentation.

### Example 1—Treatment Outcomes Using DeltaRex-G as Monotherapy for Chemoresistant Solid Malignancies

TABLE 1
Clinical trial NCT#, site, principal 1 investigator/s, phase of trial, cancer type and
treatment outcome using DeltaRex-G as monotherapy for chemoresistant solid
malignancies.

| | Clinical Site: | | | |
|---|---|---|---|---|
| | Principal | | | |
| Clinical Trial NCT # | Investigator/s | | # | |
| (Reference #) | Phase | Cancer Type | Patients | Treatment Outcome |
| NCT00121745 | Rochester MN: | Pancreatic | 12 | RECIST v1.0: 1 SD, 11 PD |
| Dose Level: minus | E. Galanis | Adenocarcinoma, | | 0% One-Year OS |
| 3-minus 1 (11) | Phase 1 | gemcitabine | | |
| | | resistant | | |
| NCT00504998* | Santa Monica CA: | Pancreatic | 20 | RECIST v1.0: 1CR, 2 PR, |
| Dose Level 1-3 | SP Chawla | Adenocarcinoma, | | 12 SD |
| (12-15) | Manhattan NY: | gemcitabine | | 28.6% One Year OS |
| | HW Bruckner | resistant | | 21.4% 1.5-Year OS |
| | (Duke) Durham | | | 1 alive in sustained |
| | NC: | | | remission, 10 years |
| | MA Morse | | | N.B.: Gained orphan |
| | Phase 1/2 | | | drug and fast track |
| | | | | designation for |
| | | | | pancreatic |
| | | | | adenocarcinoma from |
| | | | | the USFDA |
| NCT00505713* | Santa Monica CA: | Bone and Soft | 36 | 38.5% One-Year OS; |
| Dose Level 1-4 | SP Chawla, PI | Tissue Sarcoma, | | 31% 2-Year OS |
| (10, 16-17) | Phase 1/2 | Chemotherapy | | 2 alive, with no active |
| | | resistant | | disease, 10 years; |
| | | | | NB: Gained orphan drug |
| | | | | designation for soft |
| | | | | tissue |
| | | | | sarcoma from the |
| | | | | USFDA |
| NCT00505271* | Santa Monica CA: | Breast Cancer, | 20 | 60% One-Year OS |
| Dose Level 1-4 | SP Chawla, PI | chemotherapy | | |
| (unpublished | Manhattan NY: | resistant | | |
| data) | HW Bruckner, PI | | | |
| | Phase 1/2 | | | |
| NCT00572130* | Santa Monica CA: | Osteosarcoma, | 22 | 27.3% One Year OS |
| Dose Level 1-2 | SP Chawla, PI | chemotherapy | | 22.7% 2-Year OS |
| (16) | Phase 2 | resistant | | 1 alive in sustained |
| | | | | remission, 10 years; |
| | | | | N.B.: Gained orphan |
| | | | | drug |
| | | | | designation for |
| | | | | osteosarcoma from the |
| | | | | USFDA |

*Dose Level 1 = 1 × 10e11 cfu 2-3 × a week; Dose Level 2 = 2 × 10e11 cfu 3 × a wk; Dose Level 3 = 3 × 10e11 cfu 3 × a wk; Dose Level 4 = 4 × 10e11 cfu 3 × a wk; cfu = colony forming units;
OS = overall survival; CR = complete remission; PR = partial response; SD = stable disease

### Example 2—Validation of DeltaRex-G (Tumoricidal Therapy)+DeltaVax (Immunotherapy)

Ninety-nine patients received >5,000 intravenous infusions of DeltaRex-G; another 16 patients received 288 intravenous infusions of DeltaRex-G+96 infusions of DeltaVax followed by valacyclovir p.o. No therapy-related bone marrow suppression, organ dysfunction or delayed treatment related adverse events were observed. Survival analysis showed 5.0% 10-year overall survival rate for patients who received DeltaRex-G alone, and 18.8% for DeltaRex-G+DeltaVax.

Table 1 lists the cancer type, name of targeted gene therapy/immunotherapy, treatment outcome, and reference source.

| | Targeted Gene | | |
|---|---|---|---|
| | Therapy, Route of | | |
| Cancer Type | Delivery | Treatment Outcome | Reference |
| Pancreatic | DeltaRex-G, | Alive, >10 years, no evidence | Chawla et al., |
| adenocarcinoma, | intravenous | of disease, no further cancer | 2019; personal |
| metastatic to lymph | | therapy | communication |
| node, liver and | | | |
| peritoneum, resistant | | | |
| to gemcitabine and 5FU | | | |
| Osteosarcoma, | DeltaRex-G, | Alive, >10 years, no evidence | Al-Shihabi et |
| metastatic to lung, | intravenous | of disease, no further cancer | al., 2018; |
| chemotherapy- | | therapy | Chawla et al., |
| resistant | | | 2016; personal |
| | | | communication |
| Malignant peripheral | DeltaRex-G, | Alive, >10 years, no evidence | Al-Shihabi et |
| nerve sheath tumor, | intravenous | of active disease, no further | al., 2018; Kim |
| metastatic to lung, | | cancer therapy | et al., 2017; |
| chemotherapy- | | | personal |
| resistant | | | communication |
| Osteosarcoma, | DeltaRex-G, | Alive, >10 years, no evidence | Al-Shihabi et |
| metastatic to lung, | intravenous | of disease, no further cancer | al., 2018; |
| lymph nodes, pelvic | | therapy | personal |
| soft tissue, | | | communication |
| chemotherapy-resistant | | | |
| Intraductal carcinoma | DeltaRex-G, | Alive, >10 years, no evidence | Bruckner et al., |
| of breast, recurrent, | intravenous | of active disease, no further | 2019; personal |
| chemotherapy-resistant | | cancer therapy | communication |
| B-cell lymphoma, | DeltaRex-G + | Alive, 10 years, no evidence | Ignacio et al., |
| cervical lymph node, | DeltaVax, intravenous | of disease, no further cancer | 2018; personal |
| metastatic to liver and | | therapy | communication |
| pancreas, | | | |
| chemotherapy-resistant | | | |
| Chondroblastic | DeltaRex-G + | Alive, 10 years, no evidence | Ignacio et al., |
| osteosarcoma of | DeltaVax, intravenous | of active disease, no further | 2018; personal |
| maxilla, locally | | cancer therapy | communication |
| advanced, unresectable | | | |
| Intraductal carcinoma | DeltaRex-G + | Alive, >10 years, with disease | Ignacio et al., |
| of breast metastatic to | DeltaVax, intravenous | progression, on capecitabine | 2018; personal |
| bone, chemotherapy | | for liver metastases/no late | communication |
| resistant | | adverse event | |

Conclusion: Data analysis indicates that tumor-targeted gene delivery in vivo, represented by cytocidal DeltaRex-G, with or without immuno-stimulatory DeltaVax, has induced prolonged (>10 years) sustained remissions in cancer patients presenting with advanced chemotherapy-resistant solid and hematologic malignancies—plausibly due to safety, selectivity, and immune modulation. While the curative potential of precision targeted genetic medicine necessarily remains an academic question, it is clear that these initial long-term, cancer-free (>10 year) survivors represent a major milestone in both cancer therapy and immunotherapy. Phase 2-3 clinical trials are planned for these hard-to treat malignancies.

### Example 3—Transdermal Delivery of Bioactive Agents to Palpable Sarcomas

Sarc Balm 1, which contains Artemisinin plus T-Resveratrol, was applied topically, slowly, liberally over a tumor nodule. After 2 hours, Sarc Balm 2, which contains the pentacyclic triterpenes betulinuc acid, oleanoliic acid, and Boswellic acid, was applied topically, slowly, liberally over tumor nodule. These applications were repeated twice a day for three days.

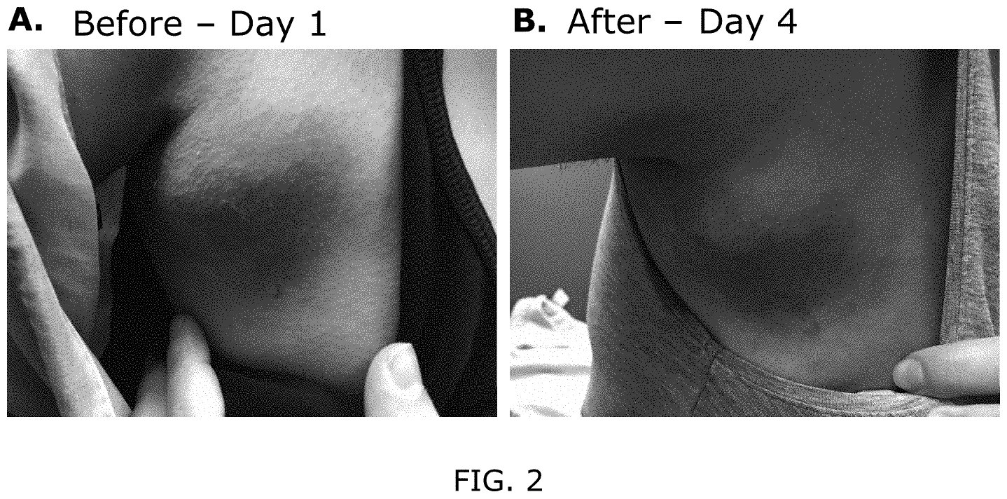

FIG. 2 provides an example of Tumor-Response Sensitivity with defined reagents in a clinical setting. These results are beneficial for both the cancer patient and the clinical oncologist, who now has a window into the disease process and its molecular genetics. This tumor's sensitivity to these bioactive agents provide a good indication that oncogenic drivers are operating and that administration of Delta RexG may be beneficial.

### REFERENCES

All references cited in this disclosure are incorporated by reference in their entirety.
1. Erlinda M. Gordon, Joshua R. Ravicz, Seiya Liu, Sant P. Chawla and Frederick L. Hall, Cell cycle checkpoint control: The cyclin G1/Mdm2/p53 axis emerges as a strategic target for broad-spectrum cancer gene therapy—A review of molecular mechanisms for oncologists, Molecular and Clinical Oncology: 2018, 9: 115-134.
2. Piette J, Neel H and Maréchal V: Mdm2: Keeping p53 under control. Oncogene 1997, 15:1001-1010.
3. Momand J, Jung D, Wilczynski S and Niland J: The MDM2 gene amplification database.

Nucleic Acids 1998, Res 26: 3453-3459.
4. Momand J, Zambetti G P, Olson D C, George D and Levine A J: The mdm-2 oncogene product forms a complex with the p53 protein and inhibits p53-mediated transactivation. Cell 1992, 69: 1237-1245.
5. Gramantieri L, Ferracin M, Fornari F, Veronese A, Sabbioni S, Liu C G, Calin G A, Giovannini C, Ferrazzi E, Grazi L G, et al: Cyclin G1 is a target of miR-122a, a microRNA frequently down-regulated in human hepatocellular carcinoma. Cancer 2007, Res 67: 6092-6099.
6. Bandopadhyay M, Sarkar N, Datta S, Das D, Pal A, Panigrahil R, Banerjee A, Panda C K, Das C, Chakrabarti S and Chakravarty R: Hepatitis B virus X protein mediated suppression of miRNA-122 expression enhances hepatoblastoma cell proliferation through cyclin G1-p53 axis. Infect Agent Cancer 2016, 11: 40.
7. Seo H R, Kim J, Bae S, Soh J W and Lee Y S: Cdk5-mediated phosphorylation of c-Myc on Ser-62 is essential in transcrip-tional activation of cyclin B1 by Cyclin G1. J Biol Chem 2008, 283: 15601-15610.
8. Menssen A and Hermeking H: Characterization of the c-MYC-regulated transcriptome by SAGE: Identification and analysis of c-MYC target genes. Proc Natl Acad Sci USA 2002, 99: 6274-6279.
9. Fornari F, Gramantieri L, Giovannini C, Veronese A, Ferracin M, Sabbioni S, Calin G A, Grazi G L, Croce C M, Tavolari S, et al: MiR-122/cyclin G1 interaction modulates p53 activity and affects doxorubicin sensitivity of human hepatocarcinoma cells. Cancer Res 2009, 69: 5761-5767.
10. Kim S, Federman N, Gordon E M, Hall F L and Chawla S P: Rexin-G®, a tumor-targeted retrovector for malignant peripheral nerve sheath tumor: A case report. Mol Clin Oncol 2017, 6: 861-865.
11. Galanis E, Carlson S K, Foster N R, Lowe V, Quevedo F, McWilliams R R, Grothey A, Jatoi A, Alberts S R and Rubin J: Phase I trial of a pathotropic retroviral vector expressing a cytocidal cyclin G1 construct (Rexin-G) in patients with advanced pancreatic cancer. Mol Ther 2008, 16: 979-984. https://doi.org/10.1038/mt.2008.29 PMID:18388964
12. Chawla S P, Chua V S, Fernandez L, Saralou A, Quon D, Blackwelder W B, Gordon E M and Hall F L: Advanced phase I/II studies of targeted gene delivery in vivo: Intravenous Rexin-G for gemcitabine-resistant metastatic pancreatic cancer. Mol Ther 2010; 18:435-441. doi:10.1038/mt2009.228.
13. Hall F L, Levy J P, Reed R A, Petchpud W N, Chua V S, Chawla S P and Gordon E M: Pathotropic targeting advances clinical oncology: Tumor-targeted localization of therapeutic gene delivery. Oncol Rep 2010, 24:829-833.
14. Bruckner H, Chawla S P, Chua V S, Quon D V, Fernandez L, Saralou A, Fisher M, Gordon E M, and Hall F L. J Clin Oncol 2010, 28:15_suppl, 4149-4149.
15. Chawla S P, Bruckner H, Morse M A, Assudani N, Hall F L, Gordon E M: A Phase I/II study on the safety and efficacy of intravenous Rexin-G, a tumor-targeted retrovector encoding a CCNG1 inhibitor for advanced pancreatic cancer. Submitted 2018.
16. Chawla S P, Chua V S, Fernandez L, Saralou A, Quon D, Blackwelder W B, Hall F L, Gordon E M: Phase I/II and Phase II studies of targeted gene delivery in vivo: intravenous Rexin-G for chemotherapy-resistant osteosarcoma and other sarcomas. Mol Ther 2009, 17:1651-1657.
17. Chawla S P, Chawla N S, Quon D, Chua-Alcala V, Blackwelder W C, Hall F L, and Gordon E M: An advanced phase 1/2 Study using an XC-targeted gene therapy vector for chemotherapy resistant sarcoma. Sarcoma Res-Int' 2016, 3: 1-7 id1024.
18. Ravicz J, Liu S, Andrali S S, Reddy S, Sellappan S, Leong B, Chawla S P, Hall F L, Gordon E M. Differential expression of human cyclin G1 (CCNG1) in cancer, a novel biomarker in development for CCNG1 inhibitor therapy. J Clin Oncol 2018, 36 (suppl; abstr e24315).
19. David E. Gerber, Targeted Therapies: A New Generation of Cancer Treatments, Am Fam Physician. 2008 Feb. 1; 77(3):311-319.
20. David E. Uehling, Philip A. Harris, Recent progress on MAP kinase pathway inhibitors, 2015, Volume 25, Issue 19, Pages 4047-4056.
21. Janice Lu. Palbociclib: a first-in-class CDK4/CDK6 inhibitor for the treatment of hormone-receptor positive advanced breast cancer. Journal of Hematology & Oncology, 2015, 8:98.
22. V M Richon. Cancer biology: mechanism of anti-tumour action of vorinostat (suberoylanilide hydroxamic acid), a novel histone deacetylase inhibitor. Br J Cancer. 2006 December; 95 (Suppl 1): S2-S6.
23. WilliamG Rice, Jeff Lightfoot, Hongying Zhang, Tiffany Cheng, Avanish Vellanki, Robert Peralta, Stephen B Howell, Andrea Local, Fannie Chau and Luis Esquivies. Clinical Pharmacokinetics of Apto-253 Support Its Use as a novel agent for the treatment of relapsed or refractory hematologic malignancies. Blood 2015 126:4934.
24. Stendahl Dy P, Chawla S P, Hall F L and Gordon E M: Immune cell trafficking in the tumor microenvironment of human cyclin G1 (CCNG1) inhibitor-treated tumors. Brit J Cancer Res 2018, 1(4): 202-207. doi: 10.31488/bjcr.117.
25. Ignacio J G, Bruckner H, Manalo R E, San Juan F S, Baniqued L, Madamba A, Gordon E M and Hall F L. Tumor-targeted cancer vaccination (GeneVieve Protocol): A phase I/II study of intravenous Rexin-G and Reximmune-C for chemotherapy-resistant cancers. J Clin Oncol 2011, 29: 2011 (suppl; abstr 2589).
26. Ignacio J G, San Juan F, Manalo R A, Soheili Nategh E, Tamhane J, Kantamneni L, et al. The Genevieve Protocol: Phase I/II evaluation of a dual targeted approach to cancer gene therapy/immunotherapy. Clin Oncol 2018, 3: 1537.
27. Efferth, T. Cancer combination therapies with artemisinin-type drugs. Biochemical Pharmacology 139 (2017) 56-70.
28. Rios, J L and Manez, S. New Pharmacological Opportunities for Betulinic Acid. Planta Med 2018; 84: 8-19.
29. Chintharlapalli S, Papineni S, Ramaiah, S K and Stephen Safe, S. Betulinic Acid Inhibits Prostate Cancer Growth through Inhibition of Specificity Protein Transcription Factors. Cancer Res 2007; 67: (6). Mar. 15, 2007.
30. Zhao X, Liu, M and Li, D. Oleanolic acid suppresses the proliferation of lung carcinoma cells by miR-122/Cyclin G1/MEF2D axis. Mol Cell Biochem. 2015 400:1-7.
31. Chen J, Huang X, Tao C, Xiao T, Li X, Zeng Q, Ma M, and Wu X. Artemether attenuates the progression of non-small cell lung cancer by inducing apoptosis, cell cycle arrest and promoting cellular senescence. Biological and Pharmaceutical Bulletin Advance Publication by J-STAGE DOI:10.1248/bpb.b19-00391. Aug. 3, 2019.

1. A method of treating a patient having an advanced metastatic cancer, the method comprising administering a plurality of infusions of a first therapeutic agent comprising a tumor-targeted gene vector that encodes a cytocidal inhibitor of the CCNG1 gene product; and administering a second therapeutic agent that affects the activity of at least one additional molecular target along the CCNG1 pathway.

2. The method of claim 1, wherein the additional molecular target is selected from the group consisting of the Mdm2, PP2A, p53, Rb and c-Myc gene products.

3. The method of claim 1 or 2, wherein the additional molecular target is an Mdm2 gene product.

4. The method of claim 3, wherein the second therapeutic agent comprises AMG232.

5. The method of claim 3, wherein the second therapeutic agent comprises Nutlin 3a.

6. The method of claim 1 or 2, wherein the additional molecular target is a PP2A gene product.

7. The method of claim 1 or 2, wherein the additional molecular target is a p53 gene product.

8. The method of claim 8, where in the second therapeutic agent comprises SAHA (vorinostat).

9. The method of claim 1 or 2, wherein the additional molecular target is an Rb gene product.

10. The method of claim 1 or 2, wherein the additional molecular target is a c-Myc gene product.

11. The method of claim 10, wherein the second therapeutic agent is APTO-253.

12. The method of claim 1, wherein the additional molecular target comprises one or more cyclin-dependent kinases.

13. The method of claim 12, wherein the second therapeutic agent comprises palbociclib.

14. The method of any of claims 1-13, wherein the first therapeutic agent comprises Delta Rex-G.

15. The method of claim 1, wherein the second therapeutic agent comprises tumor-targeted gene vector that encodes at least one tumor suppressor gene.

16. The method of claim 1, wherein the second therapeutic agent modifies RNA levels.

17. The method of claim 16, wherein the second therapeutic agent is selected from the group consisting of an antisense oligonucleotide, an RNAi construct, and a ribozyme.

18. The method of any of claims 15-17, wherein the first therapeutic agent comprises Delta Rex-G.

19. A method of treating a patient having an advanced metastatic cancer, the method comprising administering a plurality of infusions of a first therapeutic agent comprising a tumor-targeted gene vector that encodes a cytocidal inhibitor of the CCNG1 gene product; and administering a second therapeutic agent that is selected from the group consisting of immune-modulatory monoclonal antibodies, cytotoxic chemotherapies, anti-angiogenesis agents, selective tyrosine kinase inhibitors, and monoclonal antibodies directed against specific features of cells from the metastatic cancer.

20. The method of claim 19, wherein the second therapeutic agent comprises an immune-modulatory monoclonal antibody.

21. The method of claim 20, wherein the second therapeutic agent comprises a checkpoint inhibitor.

22. The method of claim 19, wherein the second therapeutic agent comprises a cytotoxic chemotherapy.

23. The method of claim 22, wherein the second therapeutic agent is selected from the group consisting of doxorubicin and trabectedin.

24. The method of claim 19, wherein the second therapeutic agent comprises an anti-angiogenesis agent.

25. The method of claim 24, wherein the second therapeutic agent comprises bevacizumab.

26. The method of claim 19, wherein the second therapeutic agent comprises a selective tyrosine kinase inhibitor.

27. The method of claim 19, wherein the second therapeutic agent comprises a monoclonal antibody directed against specific features of cells from the metastatic cancer.

28. The method of claim 27, wherein the second therapeutic agent is selected from the group consisting of panitumumab and cetuximab.

29. The method of any of claims 19-28, wherein the first therapeutic agent comprises Delta Rex-G.

30. A method of treating a palpable tumor in a patient comprising: (1) administering a plurality of infusions of a first therapeutic agent comprising a tumor-targeted gene vector that encodes a cytocidal inhibitor of the CCNG1 gene product; followed by (2) transdermally administering a second therapeutic agent comprising a ferroptosis-inducing-agent; and (3) thereafter transdermally administering a third therapeutic agent comprising an apoptosis-inducing agent.

31. The method of claim 30, wherein the third therapeutic agent is selected from a plurality of apoptosis-inducing agents after determining response sensitivity of the tumor, following administration of the first and second therapeutic agents, to the apoptosis-inducing agents and selecting at least one apoptosis-inducing agent to which the tumor is sensitive.

32. The method of claim 30 or 31, wherein the first therapeutic agent comprises DeltaRex-G.

33. The method of claim 30 or 31, wherein the ferroptosis-inducing-agent is selected from the group consisting of Artemisinin, Trans-Resveratrol, and combinations thereof.

34. The method of claim 33, wherein the first therapeutic agent comprises DeltaRex-G.

35. The method of claim 30 or 31, wherein the apoptosis-inducing agent comprises a pentacyclic triterpine.

36. The method of claim 35, wherein the pentacyclic triterpine is selected from the group consisting of betulinic acid, oleanolic acid, and boswellic acid, and combinations thereof.

37. The method of claim 35 or 36, wherein the first therapeutic agent comprises DeltaRex-G.

38. A method for evaluating the role of oncogenic drivers along the Cyclin G1 pathway in a tumor in a patient comprising determining the sensitivity of the tumor to at least one bioactive agent selected from the group consisting of ferroptosis-inducing agents, apoptosis-inducing agents, and combinations thereof by (a) administering the bioactive agent to the tumor and (b) evaluating any effects on a characteristic of the tumor; wherein tumor sensitivity to the at least one bioactive agent indicates the operation of oncogenic drivers in the tumor.

39. The method of claim 38, wherein the tumor is palpable and biotactive agents are administered transdermally.

40. The method of claim 38 or 39, wherein the bioactive agent is a pentacyclic triterpine.

41. The method of claim 40, wherein the pentacyclic triterpine is selected from the group consisting of betulinic acid, oleanolic acid, and boswellic acid, and combinations thereof.

42. A method of treating a tumor in a patient comprising: (1) evaluating the role of oncogenic drivers along the Cyclin G1 pathway in the tumor according to the method of any claims 38-41; and (2) if the tumor sensitivity to the at least one bioactive agent indicates the operation of oncogenic drivers in the tumor, administering a plurality of infusions of a first therapeutic agent comprising a tumor-targeted gene vector that encodes a cytocidal inhibitor of the CCNG1 gene product to the patient.

43. The method of claim 42, further comprising the step of administering a second therapeutic agent that affects the activity of at least one additional molecular target along the CCNG1 pathway.

44. The method of claim 43, wherein the additional molecular target is selected from the group consisting of the Mdm2, PP2A, p53, Rb and c-Myc gene products.

45. The method of claim 43 or 44, wherein the additional molecular target is an Mdm2 gene product.

46. The method of claim 45, wherein the second therapeutic agent comprises AMG232.

47. The method of claim 45, wherein the second therapeutic agent comprises Nutlin 3a.

48. The method of claim 43 or 44, wherein the additional molecular target is a PP2A gene product.

49. The method of claim 43 or 44, wherein the additional molecular target is a p53 gene product.

50. The method of claim 49, where in the second therapeutic agent comprises SAHA (vorinostat).

51. The method of claim 43 or 44, wherein the additional molecular target is an Rb gene product.

52. The method of claim 43 or 44, wherein the additional molecular target is a c-Myc gene product.

53. The method of claim 52, wherein the second therapeutic agent is APTO-253.

54. The method of claim 43, wherein the additional molecular target comprises one or more cyclin-dependent kinases.

55. The method of claim 54, wherein the second therapeutic agent comprises palbociclib.

56. The method of any of claims 42-55, wherein the first therapeutic agent comprises Delta Rex-G.

57. The method of claim 43, wherein the second therapeutic agent comprises tumor-targeted gene vector that encodes at least one tumor suppressor gene.

58. The method of claim 43, wherein the second therapeutic agent modifies RNA levels.

59. The method of claim 58, wherein the second therapeutic agent is selected from the group consisting of an antisense oligonucleotide and an RNAi construct.

60. The method of any of claims 57-59, wherein the first therapeutic agent comprises Delta Rex-G.
